(19) United States
(12) Patent Application Publication
(10) Pub. No.: US 2020/0205691 A1
(43) Pub. Date: Jul. 2, 2020

(54) PHYSIOLOGICAL SIGNAL SENSOR AND METHOD THEREOF

(71) Applicant: INDUSTRIAL TECHNOLOGY RESEARCH INSTITUTE, HSIN-CHU (TW)

(72) Inventor: YI-HSIN YU, Hsinchu City (TW)

(21) Appl. No.: 16/232,740

(22) Filed: Dec. 26, 2018

(51) Int. Cl. A61B 5/0492; A61B 5/00

(52) U.S. Cl. CPC A61B 5/0492; A61B 5/02444; A61B 5/6843

(57) ABSTRACT

A physiological signal sensor is provided, which includes an electrode layer, a pressure detection layer and a controller. The electrode layer is disposed on the skin of a subject to detect a physiological signal therefrom. The pressure detection layer detects the pressure signal of the electrode layer. The controller is connected to the electrode layer and the pressure detection layer; the controller outputs the physiological signal and outputs a measurement prompt signal according to the pressure signal.

### TECHNICAL FIELD

The technical field relates to a physiological signal sensor, in particular to a physiological signal sensor capable of determining whether the physiological signals are interfered by noises. The technical field further relates to the physiological signal detection method of the physiological signal sensor.

### BACKGROUND

A physiological signal sensor can detect the physiological signal of a subject in order to estimate the subject's healthy statuses. The physiological signal may be electrodermal activity (EDA) signal or electromyography (EMG) signal, etc. In general, the physiological signal sensor includes a dry electrode; the physiological signal sensor should be tied to the skin of the subject and then the physiological signal of the subject can be displayed by a measuring instrument. Currently, various physiological signal sensors have been developed in order to meet the requirements of different medical applications.

### SUMMARY

An embodiment of the disclosure provides a physiological signal sensor, which includes an electrode layer, a pressure detection layer and a controller. The electrode layer is disposed on a skin of a subject to detect a physiological signal therefrom. The pressure detection layer detects the pressure signal of the electrode layer. The controller is connected to the electrode layer and the pressure detection layer; the controller outputs the physiological signal and outputs a measurement prompt signal according to the pressure signal.

Another embodiment of the disclosure provides a physiological signal sensor, which includes an electrode layer, a piezoelectric detection layer and a controller. The electrode layer is disposed on the skin of a subject to detect a physiological signal therefrom. The piezoelectric detection layer detects the piezoelectric signal of the electrode layer. The controller is connected to the electrode layer and the piezoelectric detection layer. The controller outputs the physiological signal and outputs a measurement prompt signal according to the piezoelectric signal.

Still another embodiment of the disclosure provides a method for detecting physiological signal, which includes the following steps: detecting the physiological signal of the skin of a subject by an electrode layer; detecting the pressure signal of the electrode layer by a pressure detection layer; detecting the piezoelectric signal of the electrode layer by a piezoelectric detection layer; and outputting the physiological signal and outputting a measurement prompt signal according to the pressure signal and the piezoelectric signal by a controller.

Further scope of applicability of the application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure will become more fully understood from the detailed description given herein below and the accompanying drawings which are given by way of illustration only, and thus are not limitative of the disclosure and wherein:

FIG. 1 is a structure diagram of a physiological signal sensor in accordance with a first embodiment of the disclosure.

FIG. 2 is a flow chart of the first embodiment.

FIG. 3 is a structure diagram of a physiological signal sensor in accordance with a second embodiment of the disclosure.

FIG. 4 is a flow chart of the second embodiment.

FIG. 5 is a structure diagram of a physiological signal sensor in accordance with a third embodiment of the disclosure.

FIG. 6 is a flow chart of the third embodiment.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

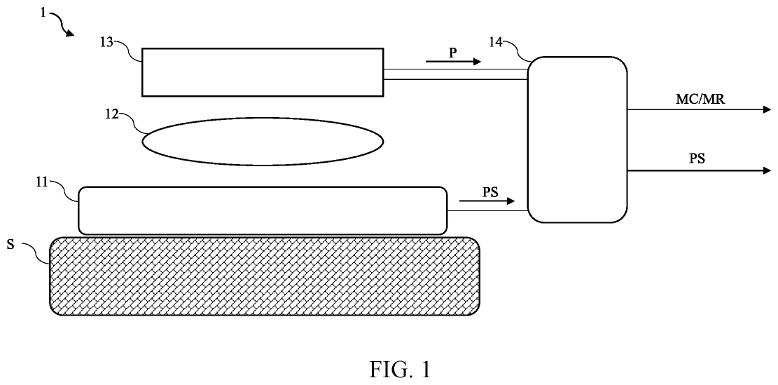

FIG. 1 is a structure diagram of a physiological signal sensor in accordance with a first embodiment of the disclosure. As shown in FIG. 1, the physiological signal sensor 1 includes an electrode layer 11, a first insulating layer 12, a pressure detection layer 13 and a controller 14.

The electrode layer 11 is disposed on the skin S of a subject to detect the physiological signal PS therefrom; the electrode layer 11 may be made of a flexible, bendable and stretchable material. In one embodiment, the electrode layer 11 may be made of a Si—Ag based material. In one embodiment, the physiological signal PS may be electrodermal activity (EDA) signal or electromyography (EMG) signal, etc.

The pressure detection layer 13 detects the pressure signal P of the electrode layer 11, which can serve as the auxiliary signal for determining the reliability of the physiological signal PS. In one embodiment, the pressure detection layer 13 may be made of a piezo resistive material or a piezoelectric signal, etc.

The first insulating layer 12 is disposed between the electrode layer 11 and the pressure detection layer 13 to insulate the electrode layer 11 from the pressure detection layer 11. In one embodiment, the first insulating layer 12 may be made of plastics.

The controller 14 is connected to the electrode layer 11 and the pressure detection layer 13, and outputs the physiological signal PS. In one embodiment, the controller 14 may be a microcontroller unit (MCU) or other similar devices.

The controller 14 can determine the reliability of the physiological signal PS (i.e. whether the physiological signal PE is detected when the electrode layer 11 actually contacts the skin S), generates a measurement prompt signal according to the pressure signal, and then outputs the physiological signal PS and the measurement prompt signal. When the pressure signal P is within a predetermined range, the controller 14 determines that the electrode layer 11 contacts the skin S. Then, the controller 14 outputs the physiological signal PS and a correct measurement signal MC. On the contrary, when the pressure signal P is not within the predetermined range, the controller 14 determines that the electrode layer 11 does not contact the skin S. Afterward, the controller 14 outputs the physiological signal PS and an incorrect measurement signal MR. Different test items may have different test ranges; therefore, the tester can determine the above predetermined range according to the test conditions of the test items. In one embodiment, the above predetermined range may be 0.098 N˜4 N (Newton). In another embodiment, the above predetermined range may be 4 N˜6 N. In still another embodiment, the above predetermined range may be 6˜10 N.

Via the above mechanism, the controller 14 can effectively determine whether the electrode layer 11 of the physiological signal sensor 1 falls off or contacts the skin

S, and generates an incorrect measurement signal MR when the electrode layer 11 does not contact the skin S. In this way, the tester can understand whether the physiological signal PS is correctly detected, so the precision of the physiological signal measurement can be effectively enhanced.

In addition, the electrode layer 11 may be made of a Si—Ag based material, which is a flexible, bendable and stretchable material, and the impedance thereof is lower than 10 Ω. Thus, the electrode layer 11 can effectively improve the precision of the physiological signal PS and the subject can also comfortably wear the physiological signal sensor 1.

The embodiment just exemplifies the disclosure and is not intended to limit the scope of the disclosure. Any equivalent modification and variation according to the spirit of the disclosure is to be also included within the scope of the following claims and their equivalents.

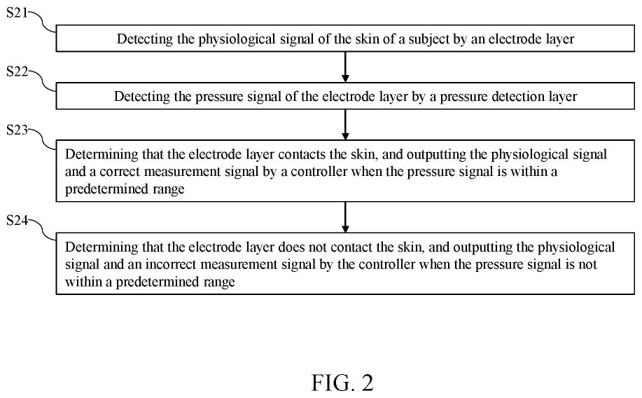

Please refer to FIG. 2, which is a flow chart of the first embodiment. As shown in FIG. 2, the physiological signal detection method of the physiological signal sensor 1 in accordance with the first embodiment includes the following steps:

Step S21: detecting the physiological signal of the skin of a subject by an electrode layer.

Step S22: detecting the pressure signal of the electrode layer by a pressure detection layer.

Step S23: determining that the electrode layer contacts the skin, and outputting the physiological signal and a correct measurement signal by a controller when the pressure signal is within a predetermined range.

Step S24: determining that the electrode layer does not contact the skin, and outputting the physiological signal and an incorrect measurement signal by the controller when the pressure signal is not within a predetermined range.

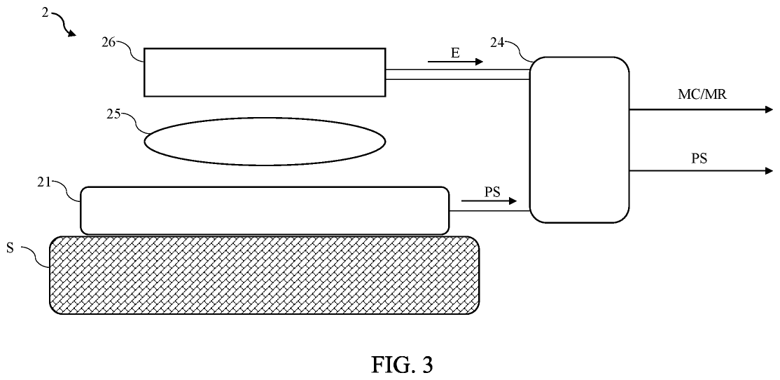

FIG. 3 is a structure diagram of a physiological signal sensor in accordance with a second embodiment of the disclosure. As shown in FIG. 3, the physiological signal sensor 2 includes an electrode layer 21, a second insulating layer 25, a piezoelectric detection layer 26 and a controller 24.

The electrode layer 21 is disposed on the skin S of a subject to detect the physiological signal PS therefrom.

The piezoelectric detection layer 26 detects the piezoelectric signal E of the electrode layer 21, which can serve as the auxiliary signal for determining the reliability of the physiological signal PS. In one embodiment, the piezoelectric detection layer 26 may be made of a piezo resistive material or a piezoelectric signal, etc.

The second insulating layer 25 is disposed between the electrode layer 21 and the piezoelectric detection layer 26 to insulate the electrode layer 21 from the piezoelectric detection layer 26. In one embodiment, the second insulating layer 25 may be made of plastics.

The controller 14 is connected to the electrode layer 11 and the piezoelectric detection layer 26, and outputs the physiological signal PS.

The controller 24 can determine the reliability of the physiological signal PS (i.e. whether the physiological signal PS is interfered by noises), generates a measurement prompt signal according to the piezoelectric signal E, and then outputs the physiological signal PS and the measurement prompt signal. When the piezoelectric signal E shows that the piezoelectric detection layer 26 is uniformly deformed, the controller 14 determines that the external force applied to the electrode layer 21 is stable. Afterwards, the controller 14 outputs the physiological signal PS and a correct measurement signal MC. On the contrary, when the piezoelectric signal E shows that the piezoelectric detection layer 26 is non-uniformly deformed, the controller 14 determines that the external force applied to the electrode layer 21 is unstable. Then, the controller 14 outputs the physiological signal PS and an incorrect measurement signal MR.

Via the above mechanism, the controller 14 can effectively determine whether the electrode layer 21 of the physiological signal sensor 2 is properly fixed on the skin S and whether the electrode layer 21 is interfered by noises generated by improper external force. In this way, the tester can understand whether the physiological signal PS is correctly detected, so the precision of the physiological signal measurement can be effectively enhanced.

The embodiment just exemplifies the disclosure and is not intended to limit the scope of the disclosure. Any equivalent modification and variation according to the spirit of the disclosure is to be also included within the scope of the following claims and their equivalents.

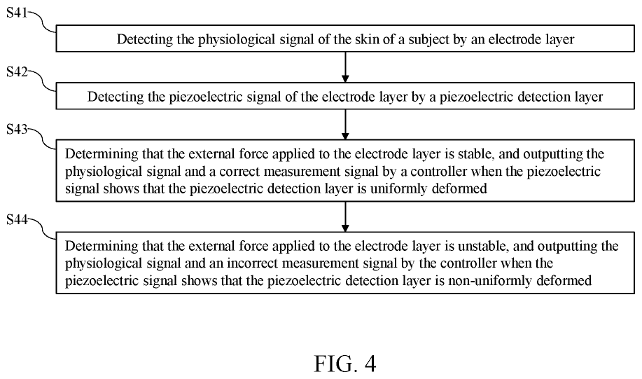

Please refer to FIG. 4, which is a flow chart of the second embodiment. As shown in FIG. 4, the physiological signal detection method of the physiological signal sensor 2 in accordance with the second embodiment includes the following steps:

Step S41: detecting the physiological signal of the skin of a subject by an electrode layer.

Step S42: detecting the piezoelectric signal of the electrode layer by a piezoelectric detection layer.

Step S43: determining that the external force applied to the electrode layer is stable, and outputting the physiological signal and a correct measurement signal by a controller when the piezoelectric signal shows that the piezoelectric detection layer is uniformly deformed.

Step S44: determining that the external force applied to the electrode layer is unstable, and outputting the physiological signal and an incorrect measurement signal by the controller when the piezoelectric signal shows that the piezoelectric detection layer is non-uniformly deformed.

It is worthy to point out that currently available dry electrodes tend to be influenced by environmental factors, so detected physiological signals may be interfered by noises. However, the currently available dry electrodes have no an effective mechanism to determine whether the detected physiological signals are interfered by noises. On the contrary, according to one embodiment of the disclosure, the physiological signal sensor includes the pressure detection layer for detecting the pressure signal of the electrode layer. The pressure signal can serve as an auxiliary signal to determine whether the electrode layer contacts the skin of the subject. In this way, the physiological signal sensor can effectively determine whether the physiological signal is correctly detected in order to enhance the accuracy of the physiological signal measurement.

In addition, the physiological signal sensor includes the piezoelectric sensing layer for detecting the piezoelectric signal of the electrode layer. The piezoelectric signal can serve as an auxiliary signal to determine whether the external force applied to the electrode layer is stable. Thus, the physiological signal sensor can effectively determine whether the physiological signal is correctly detected in order to enhance the accuracy of the physiological signal measurement.

Moreover, as the currently available dry electrodes tend to be interfered by noises, so the measurement instrument may need a complicated algorithm to filter out noises. Therefore, the currently available dry electrodes are not convenient to use and of low efficiency. On the contrary, according to one embodiment of the disclosure, the physiological signal sensor can provide the auxiliary signals for determining whether the physiological signal is correctly detected, which is more convenient to use and can achieve better efficiency.

Furthermore, according to one embodiment of the disclosure, the physiological signal sensor can be made of the flexible and stretchable Si—Ag based material, which can not only reduce the impedance of the electrode layer, but also can increase the flexibility of the electrode layer. Therefore, the subject can comfortably wear the physiological signal sensor. As described above, the physiological signal sensor according to the embodiments of the disclosure can definitely achieve unpredictable technical effects.

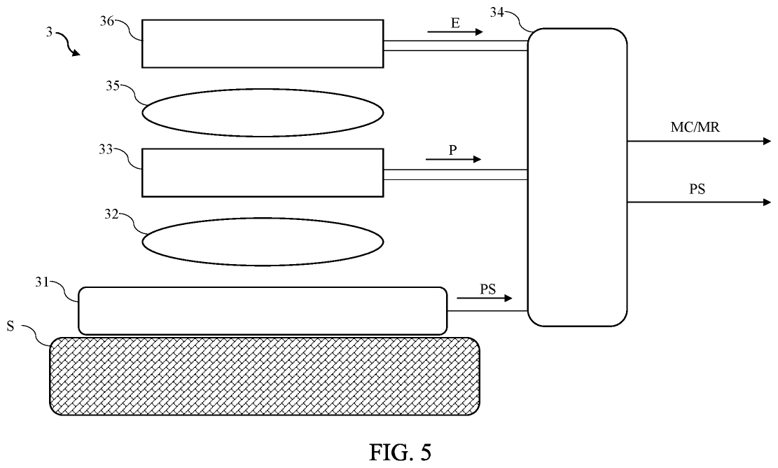

FIG. 5 is a structure diagram of a physiological signal sensor in accordance with a third embodiment of the disclosure. As shown in FIG. 5, the physiological signal sensor 3 includes an electrode layer 31, a first insulating layer 32, a pressure detection layer 33, a second insulating layer 35, a piezoelectric detection layer 36 and a controller 34.

The electrode layer 31 is disposed on the skin S of a subject to detect the physiological signal PS therefrom.

The pressure detection layer 33 detects the pressure signal P of the electrode layer 31.

The piezoelectric detection layer 26 detects the piezoelectric signal E of the electrode layer 21.

The first insulating layer 32 is disposed between the electrode layer 21 and the pressure detection layer 33 to insulate the electrode layer 21 from the pressure detection layer 33.

The second insulating layer 35 is disposed between the pressure detection layer 33 and the piezoelectric detection layer 36 to insulate the pressure detection layer 33 and the piezoelectric detection layer 36.

The controller 34 is connected to the electrode layer 31, the pressure detection layer 33 and the piezoelectric detection layer 36, and outputs the physiological signal PS.

The difference between the embodiment and the previous embodiments is that the physiological signal sensor 3 of the embodiment includes both the pressure detection layer 33 and the piezoelectric detection layer 36. Thus, the controller 24 can determine the reliability of the physiological signal PS according to the pressure signal P and the piezoelectric signal E, and then output the physiological signal PS and a measurement prompt signal.

When the pressure signal P is within a predetermined range and the piezoelectric signal E shows that the piezoelectric detection layer 36 is uniformly deformed, the controller 34 determines that the electrode layer 31 contacts the skin S and the external force applied to the electrode layer 31 is stable. Then, the controller 34 outputs the physiological signal PS and a correct measurement signal MC.

When the pressure signal P is within a predetermined range and the piezoelectric signal E shows that the piezoelectric detection layer 36 is non-uniformly deformed, the controller 34 determines that the electrode layer 31 contacts the skin S but the external force applied to the electrode layer 31 is unstable. Then, the controller 34 outputs the physiological signal PS and an incorrect measurement signal MR.

When the pressure signal P is not within a predetermined range and the piezoelectric signal E shows that the piezoelectric detection layer 36 is uniformly deformed, the controller 34 determines that the electrode layer 31 does not contact the skin S but the external force applied to the electrode layer 31 is stable. Then, the controller 34 outputs the physiological signal PS and an incorrect measurement signal MR.

When the pressure signal P is not within a predetermined range and the piezoelectric signal E shows that the piezoelectric detection layer 36 is non-uniformly deformed, the controller 34 determines that the electrode layer 31 does not contact the skin S and the external force applied to the electrode layer 31 is unstable. Then, the controller 34 outputs the physiological signal PS and an incorrect measurement signal MR.

Via the above mechanism, the controller 34 can effectively determine whether the electrode layer 31 of the physiological signal sensor 3 contacts the skin S and whether the electrode layer 21 is interfered by noises generated by improper external force. Further, the controller 34 can generate the incorrect measurement signal MR when the electrode layer 31 does not contact the skin S or is interfered by noises. In this way, the tester can understand whether the physiological signal PS is correctly detected, so the precision of the physiological signal measurement can be effectively enhanced.

The embodiment just exemplifies the disclosure and is not intended to limit the scope of the disclosure. Any equivalent modification and variation according to the spirit of the disclosure is to be also included within the scope of the following claims and their equivalents.

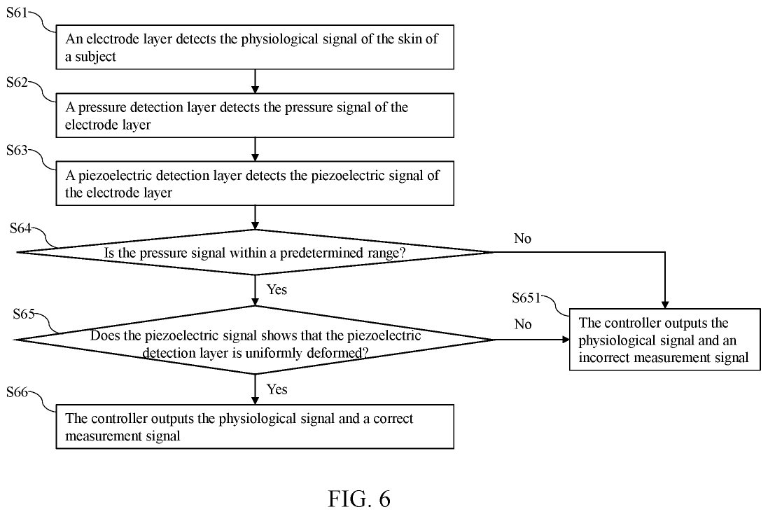

Please refer to FIG. 6, which is a flow chart of the third embodiment. As shown in FIG. 6, the physiological signal detection method of the physiological signal sensor 3 in accordance with the third embodiment includes the following steps:

Step S61: an electrode layer detects the physiological signal of the skin of a subject; then, the process proceeds to Step S62.

Step S62: detect the pressure signal of the electrode layer by a pressure detection layer; then, the process proceeds to Step S63.

Step S63: detect the piezoelectric signal of the electrode layer by a piezoelectric detection layer; then, the process proceeds to Step S64.

Step S64: a controller determines whether the pressure signal is within a predetermined range? If it is, the process proceeds to Step S65; if it is not, the process proceeds to Step S651.

Step S65: the controller determines whether the piezoelectric detection layer is uniformly deformed according to the piezoelectric signal? If it is, the process proceeds to Step S66; if it is not, the process proceeds to Step S651.

Step S651: the controller outputs the physiological signal and an incorrect measurement signal.

Step S66: the controller outputs the physiological signal and a correct measurement signal.

In summation of the description above, according to one embodiment of the disclosure, the physiological signal sensor includes the pressure detection layer for detecting the pressure signal of the electrode layer. The pressure signal can serve as an auxiliary signal to determine whether the electrode layer contacts the skin of the subject. In this way, the physiological signal sensor can effectively determine whether the physiological signal is correctly detected in order to enhance the accuracy of the physiological signal measurement.

In addition, according to one embodiment of the disclosure, the physiological signal sensor includes the piezoelectric sensing layer for detecting the piezoelectric signal of the electrode layer. The piezoelectric signal can serve as an auxiliary signal to determine whether the external force applied to the electrode layer is stable. Thus, the physiological signal sensor can effectively determine whether the physiological signal is correctly detected in order to enhance the accuracy of the physiological signal measurement.

Moreover, according to one embodiment of the disclosure, the physiological signal sensor can provide the auxiliary signals for determining whether the physiological signal is correctly detected, which is more convenient to use and can achieve better efficiency.

Furthermore, according to one embodiment of the disclosure, the physiological signal sensor can be made of the flexible and stretchable Si—Ag based material, which can not only reduce the impedance of the electrode layer, but also can increase the flexibility of the electrode layer. Therefore, the subject can comfortably wear the physiological signal sensor.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A physiological signal sensor, comprising:
an electrode layer, disposed on a skin and configured to detect a physiological signal therefrom;
a pressure detection layer, configured to detect a pressure signal of the electrode layer; and
a controller, connected to the electrode layer and the pressure detection layer, and configured to output the physiological signal and output a measurement prompt signal according to the pressure signal.

2. The physiological signal sensor of claim 1, wherein when the pressure signal is within a predetermined range, the controller determines that the electrode layer contacts the skin; when the pressure signal is not within the predetermined range, the controller determines that the electrode layer does not contact the skin.

3. The physiological signal sensor of claim 2, further comprising a piezoelectric detection layer connected to the controller, and configured to detect a piezoelectric signal of the electrode layer and determine whether the physiological signal is interfered by a noise according to the piezoelectric signal.

4. The physiological signal sensor of claim 3, wherein when the piezoelectric signal shows that the piezoelectric detection layer is uniformly deformed, the controller determines that an external force applied to the electrode layer is stable; when the piezoelectric signal shows that the piezoelectric detection layer is non-uniformly deformed, the controller determines that the external force applied to the electrode layer is unstable.

5. The physiological signal sensor of claim 3, further comprising a first insulating layer disposed between the electrode layer and the pressure detection layer.

6. The physiological signal sensor of claim 5, further comprising a second insulating layer disposed between the pressure detection layer and the piezoelectric detection layer.

7. The physiological signal sensor of claim 1, wherein the electrode layer is made of a flexible, bendable and stretchable material.

8. The physiological signal sensor of claim 1, wherein the electrode layer is made of a Si—Ag based material.

9. A physiological signal sensor, comprising:
an electrode layer, disposed on a skin and configured to detect a physiological signal therefrom;
a piezoelectric detection layer, configured to detect a piezoelectric signal of the electrode layer; and
a controller, connected to the electrode layer and the piezoelectric detection layer, and configured to output the physiological signal and output a measurement prompt signal according to the piezoelectric signal.

10. The physiological signal sensor of claim 9, wherein when the piezoelectric signal shows that the piezoelectric detection layer is uniformly deformed, the controller determines that an external force applied to the electrode layer is stable; when the piezoelectric signal shows that the piezoelectric detection layer is non-uniformly deformed, the controller determines that the external force applied to the electrode layer is unstable.

11. The physiological signal sensor of claim 10 further comprising a pressure detection layer connected to the controller and configured to detect a pressure signal of the electrode layer.

12. The physiological signal sensor of claim 11, wherein when the pressure signal is within a predetermined range, the controller determines that the electrode layer contacts the skin; when the pressure signal is not within the predetermined range, the controller determines that the electrode layer does not contact the skin.

13. The physiological signal sensor of claim 11, further comprising a first insulating layer disposed between the electrode layer and the pressure detection layer.

14. The physiological signal sensor of claim 13, further comprising a second insulating layer disposed between the pressure detection layer and the piezoelectric detection layer.

15. The physiological signal sensor of claim 9, wherein the electrode layer is made of a Si—Ag based material.

16. A method for detecting physiological signal, comprising:
Detecting a physiological signal of a skin by an electrode layer;
detecting a pressure signal of the electrode layer by a pressure detection layer;
detecting a piezoelectric signal of the electrode layer by a piezoelectric detection layer; and
outputting the physiological signal and outputting a measurement prompt signal according to the pressure signal and the piezoelectric signal by a controller.

17. The method of claim 16, wherein a step of outputting the physiological signal and outputting the measurement prompt signal according to the pressure signal and the piezoelectric signal by the controller further comprising:
determining that the electrode layer contacts the skin and an external force applied to the electrode layer is stable, and outputting the physiological signal and a correct measurement signal by the controller when the pressure signal is within a predetermined range and the piezoelectric signal shows that the piezoelectric detection layer is uniformly deformed.

18. The method of claim 16, wherein a step of outputting the physiological signal and outputting the measurement prompt signal according to the pressure signal and the piezoelectric signal by the controller further comprising:
determining that the electrode layer contacts the skin and an external force applied to the electrode layer is unstable, and outputting the physiological signal and an incorrect measurement signal by the controller when the pressure signal is within a predetermined range and the piezoelectric signal shows that the piezoelectric detection layer is non-uniformly deformed.

19. The method of claim 16, wherein a step of outputting the physiological signal and outputting the measurement prompt signal according to the pressure signal and the piezoelectric signal by the controller further comprising:
determining that the electrode layer does not contact the skin and an external force applied to the electrode layer is stable, and outputting the physiological signal and an incorrect measurement signal by the controller when the pressure signal is not within a predetermined range and the piezoelectric signal shows that the piezoelectric detection layer is uniformly deformed.

20. The method of claim 16, wherein a step of outputting the physiological signal and outputting the measurement prompt signal according to the pressure signal and the piezoelectric signal by the controller further comprising:
determining that the electrode layer does not contact the skin and an external force applied to the electrode layer is unstable, and outputting the physiological signal and an incorrect measurement signal by the controller when the pressure signal is not within a predetermined range and the piezoelectric signal shows that the piezoelectric detection layer is non-uniformly deformed.
